(19) United States
(12) Patent Application Publication
(10) Pub. No.: US 2023/0270401 A1
(43) Pub. Date: Aug. 31, 2023

(54) RADIOLOGY ASSEMBLY AND METHOD FOR ALIGNING SUCH AN ASSEMBLY

(71) Applicant: TRIXELL, MOIRANS (FR)

(72) Inventors: Fabien VERMONT, MEYLAN (FR); Olivier SOSNICKI, MEYLAN (FR); Patrick MENEROUD, VIF (FR); Bruno COMMERE, MOIRANS (FR); Laurent CHEVALLIER, MOIRANS (FR); Guillaume JOLY, MOIRANS (FR)

(21) Appl. No.: 18/024,033

(22) PCT Filed: Sep. 22, 2021
(86) PCT No.: PCT/EP2021/075992

§ 371 Date: Feb. 28, 2023

(30) Foreign Application Priority Data
Sep. 28, 2020 (FR) FR2009823

(51) Int. Cl. A61B 6/00

(52) U.S. Cl. CPC A61B 6/587

(57) ABSTRACT

A radiology assembly includes an x-ray tube for generating a beam of x-rays that is centered around a main emission direction, a planar sensor extending in a plane defined by a first direction and by a second direction, which directions are substantially perpendicular to the main x-ray emission direction, the sensor being intended to receive the x-rays, comprising a first divided emitter that is divided into two electromagnetic-field-emitting portions; a second divided emitter that is divided into two electromagnetic-field-emitting portions; a so-called planar electromagnetic-field emitter, electromagnetic-field sensors that are securely fastened to the planar sensor, a processing means intended to determine an angle of alignment between the main emission direction and a normal of the planar sensor, to determine a first centering error and a second centering error, a correcting means for correcting the angle of alignment by applying a first corrective movement to the x-ray tube and first and second centering errors by applying the first corrective movement and/or a second corrective movement to the x-ray tube.

### CROSS-REFERENCE TO RELATED APPLICATIONS

This application is a National Stage of International patent application PCT/EP2021/075992, filed on Sep. 22, 2021, which claims priority to foreign French patent application No. FR 2009823, filed on Sep. 28, 2020, the disclosures of which are incorporated by reference in their entirety.

### FIELD OF THE INVENTION

The invention relates to a radiology assembly and more precisely to the alignment of two elements of the radiology assembly, namely the planar sensor with respect to the x-ray tube. The invention also relates to a method for aligning such a radiology assembly. The invention pertains to the field of radiology (for example medical or veterinary radiology) but is not limited to this field. The invention finds a possible application in the fields of safety and industrial inspection. The invention may also be applied to other fields in which it is necessary to align a point source of radiation with a planar sensor, for example in the field of infrared imaging.

### BACKGROUND

In the present patent application, the invention is presented in a case of application to a radiology assembly. Nevertheless, the invention may find application in other fields requiring two elements to be correctly positioned with respect to each other.

A radiology assembly consists of two elements: an x-ray tube for generating a beam of x-rays and a planar sensor of radiographic images. The assembly is intended to mainly produce radiographic images of patients in a hospital setting. A patient, whom it is desired to radiograph, is placed between the x-ray tube and the planar sensor. The two elements must therefore be well-positioned with respect to each other so that all the x-rays emitted by the x-ray tube are captured by the planar sensor. The two elements are then said to be correctly aligned. The alignment must be carried out before the x-rays are emitted by the x-ray tube. The aim is to prevent the patient from being over-irradiated with x-rays that are not captured by the sensor while obtaining a good image quality.

Generally, the x-ray tube is aligned manually by an operator to face the planar sensor. The alignment is carried out translationwise and rotationwise. The alignment is generally carried out when the patient is in place, i.e. positioned between the x-ray tube and the planar sensor. There are many particular cases in which the planar sensor is masked. Mention may be made, by way of example, of the case in which the planar sensor is placed under a patient for a radiograph of the abdomen or of the pelvis. Mention may also be made of the case in which the planar sensor is placed under a sheet, under a stretcher or even in an incubator. It is therefore, in this case, very difficult for the operator to align the x-ray tube with respect to the planar sensor.

Moreover, the environment of the planar sensor may be of several types. The environment may in particular be a hospital bed or a stretcher including metal frames or an incubator for premature babies. The environment of the sensor may therefore be an additional hindrance with regard to correct positioning of the x-ray tube with respect to the planar sensor.

Alignment of the first element with respect to the second element comprises correction of several defects: centering defect (the beam of x-rays is not centered on the planar sensor), orientation defect (the beam of x-rays is poorly oriented with respect to the plane of the planar sensor) and perpendicularity defect (the beam of x-rays does not strike the planar sensor perpendicularly). The perpendicularity defect is critical when an anti-scatter grid is used to produce the image. The grid is then placed on the planar sensor. The x-rays, in order to be able to be detected by the planar sensor, must strike the sensor perpendicularly to the planar sensor. The angular tolerance with respect to perpendicularity is small (only a few degrees).

There are several ways of proceeding with alignment of two elements. Mention may firstly be made of optical alignment in which the two elements are aligned by means of light beams that measure the relative position of one element with respect to the other. Optical alignment cannot be used in the field of radiology since the planar sensor is often partially masked by a bed sheet or by the patient.

Alignment may also be achieved by means of beams of acoustic waves. However, as the alignment is carried out in the presence of the patient, the patient may mask all or some of the planar sensor. In addition, the presence of the patient may locally attenuate the acoustic waves and thus corrupt the measurement of the distance between the planar sensor and the x-ray tube.

It is also possible to carry out the alignment of two elements on the basis of the measurement of the propagation time of an electromagnetic wave. The measurement of the propagation time of a wave makes it possible to measure the distance between the two elements. By triangulation, it is possible to determine the relative position of the two elements with respect to each other. However, this alignment technique cannot be successfully used in the case of an application to radiology since the propagation time of the electromagnetic wave may vary depending on the position of the patient between the two elements (x-ray tube and planar sensor). In addition, multiple echoes may be generated because of the environment (bed, stretcher, etc.), the echoes possibly having signal levels higher than the main signal.

On the same principle, an aligning technique exists based on the measurement of the attenuation of an electromagnetic signal to measure the distance between two elements. As, in the case of an application to radiology, the patient may locally attenuate the electromagnetic wave and therefore corrupt the measurement, this alignment technique is unsuitable.

Furthermore, the patent U.S. Pat. No. 10,080,542 describes a method of providing information for aligning an x-ray tube and a detector of a mobile x-ray apparatus using sensors for sensing the orientation of an absolute position of the x-ray tube and of the detector. A single magnetic field is generated at the x-ray tube along an axis traversing the x-ray tube and the detector in order to be evaluated, by means of sensors, at the detector. The relative orientation information in terms of rotation is calculated by the difference between the absolute orientations of the x-ray tube and of the detector. Relative positioning in terms of translation is carried out by comparing the values of the measured components of the magnetic field with those that have been pre-registered. Since the information is obtained over the six degrees of mobility, the use of a single magnetic field appears insufficient in terms of robustness in the case of a measuring error of the single magnetic field. In addition, the use of a single magnetic field involves the use of complex formulae in order to determine the six degrees of mobility, thus making the alignment algorithm more complex.

Lastly, a dental radiology system (patent FR 2 899 349) uses a plurality of electromagnetic-field emitters that are placed in one and the same plane and one or two electromagnetic-field receivers that are able to receive the electromagnetic fields emitted by the emitters. The use of two receivers makes it possible to determine the angular orientation of the sensor but gives no indication as to the angle of one element with respect to the other (x-ray tube with respect to the planar sensor). In addition, the positioning of the emitters in one and the same plane gives a mediocre indication as to the location of the planar sensor with respect to the x-ray tube. It will be noted that dental radiology covers a relatively short distance (20 to 30 cm) between the x-ray tube and the sensor, compared to the distance between the x-ray tube and the sensor in the field of medical radiology (rather about 1 to 2 m).

### SUMMARY OF THE INVENTION

The invention aims to mitigate all or some of the aforementioned problems by providing a radiology assembly having a plurality of electromagnetic-field emitters that are securely fastened to the x-ray tube and are positioned in separate planes, and having a plurality of electromagnetic-field sensors that are positioned on the planar sensor and receive the x-rays. This assembly makes it possible to unambiguously know the spatial position of the planar sensor and therefore to know its position with respect to the x-ray tube, and thus to align and position the x-ray tube with respect to the planar sensor. More specifically, the invention is based on perpendicularly aligning the planar sensor with respect to the x-ray tube and then centering around the main emission direction of the x-rays.

To this end, one subject of the invention is a radiology assembly comprising:
an x-ray tube for generating a beam of x-rays that is centered around a main emission direction,
a planar sensor extending in a plane defined by a first direction and by a second direction, which directions are substantially perpendicular to the main x-ray emission direction, the sensor being intended to receive the x-rays,
   characterized in that it comprises:
a first divided emitter that is divided into two electromagnetic-field-emitting portions and is arranged so as to emit a first electromagnetic field in a main direction that is substantially perpendicular to the main emission direction, each of the two emitting portions of the divided emitter being positioned on one respective side of the beam of x-rays,
a second divided emitter that is divided into two electromagnetic-field-emitting portions and is arranged so as to emit a second electromagnetic field in a main direction that is substantially perpendicular to the main emission direction and is secant to the main direction of the first electromagnetic field, each of the two portions of the divided emitter being positioned on one respective side of the beam of x-rays,
a so-called planar electromagnetic-field emitter, the so-called planar emitter being a coil composed of windings, the so-called planar emitter being arranged so as to emit a third electromagnetic field in a main direction that is substantially parallel to the main emission direction of the beam of x-rays, the windings being passed through by the main emission direction,
electromagnetic-field sensors that are securely fastened to the planar sensor and are able to detect the first, second and third electromagnetic fields emitted alternately in their main direction by the first emitter, the second emitter and the so-called planar emitter and to generate a first, second, third electrical signal depending on the detected electromagnetic fields,
a processing means for processing the first, second and third electrical signals that is intended to determine an angle of alignment between the main emission direction and a normal of the planar sensor, to determine a first centering error between the main emission direction of the first electromagnetic field and the first direction of the planar sensor, to determine a second centering error between the main emission direction of the second electromagnetic field and the second direction of the planar sensor,
a correcting means for correcting the angle of alignment by applying a first corrective movement to the x-ray tube and first and second centering errors by applying the first corrective movement and/or a second corrective movement to the x-ray tube.

According to one aspect of the invention, the processing means comprises means for distinguishing the generated electrical signals.

According to one aspect of the invention, the processing means for processing the first, second and third electrical signals comprises an estimator for estimating an angle of orientation between the main direction of the first electromagnetic field and the first direction of the planar sensor.

According to one aspect of the invention, each of the two emitting portions of the first and second divided emitter comprises at least one winding and the main emission direction of the beam of x-rays is positioned between the at least one winding of the first and second divided emitter.

According to one aspect of the invention, the so-called planar emitter comprises at least one winding that is passed through by the main emission direction of the beam of x-rays.

According to one aspect of the invention, the two emitting portions of the first and second divider emitter and the so-called planar emitter are flat coils.

According to one aspect of the invention, the first corrective movement is a rotation of the x-ray tube in one of the main directions and/or a rotation of the x-ray tube in the main emission direction and wherein the second corrective movement is a translation of the x-ray tube in one of the main directions.

According to one aspect of the invention, the planar sensor comprises at least one inclinometer.

According to one aspect of the invention, the processing means and the correcting means are mechanically linked to the planar sensor.

According to one aspect of the invention, the processing means and the correcting means are mechanically linked to the x-ray tube.

The invention also relates to a method for aligning a radiology assembly, characterized in that it includes the following steps:
Emission by the first divided emitter of the first electromagnetic field in the main direction that is substantially perpendicular to the main emission direction,
Emission by the second emitter of the second electromagnetic field in the main direction that is substantially perpendicular to the main emission direction,
Emission by the so-called planar emitter of a third electromagnetic field in a main direction that is substantially parallel to the main emission direction,
Detection by the sensors of the electromagnetic fields emitted alternately in their main direction by the first emitter, the second emitter and the so-called planar emitter,
Generation of the first, second, third electrical signals by the sensors depending on the first, second, third detected electromagnetic fields,
Evaluation of the angle of alignment between the main emission direction and a normal of the planar sensor,
Correction of the angle of alignment between the main emission direction and a normal of the planar sensor by applying the first corrective movement,
Evaluation of the first centering error between the main emission direction of the first electromagnetic field and the first direction of the planar sensor, and of the second centering error between the main emission direction of the second electromagnetic field and the second direction of the planar sensor,
Correction of the first and the second centering error by applying the second corrective movement,
Optionally, repetition of the preceding steps until the angle of alignment is less than a predefined threshold angle of alignment and/or until the first centering error and the second centering error are less than a predefined threshold first centering error and than a predefined threshold second centering error.

According to one aspect of the invention, the method includes, beforehand, a calibration step intended to calibrate the electrical signal as a function of predetermined positions of the x-ray tube and of the planar sensor.

According to one aspect of the invention, the emission by the emitters of the electromagnetic fields includes a step of supplying the emitters with power, and in that the emitters are supplied with power at different instants or simultaneously at different frequencies or simultaneously in phase offset so as to differentiate the emitted electromagnetic fields.

According to one aspect of the invention, the method comprises a step of evaluation of the angle of orientation between the main direction of the first electromagnetic field and the first direction of the planar sensor following the step of correction of the centering error and comprises a step of correction of the angle of orientation between the main direction of the first electromagnetic field and the first direction of the planar sensor following the step of evaluation of the angle of orientation.

### BRIEF DESCRIPTION OF THE DRAWINGS

The invention will be better understood and other advantages will become apparent on reading the detailed description of one embodiment given by way of example, the description being illustrated by the attached drawing, in which:

FIG. 1 shows one embodiment of a radiology assembly according to the invention,

FIG. 2 shows an example of an arrangement of the electromagnetic-field emitters according to the invention,

FIG. 3 shows an example of a holder of the electromagnetic-field emitters,

FIG. 4 shows a cross-sectional view of a radiology assembly according to the invention,

FIG. 5 schematically shows the steps of an aligning method according to the invention.

For the sake of clarity, the same elements have been designated by the same references in the various figures.

### DETAILED DESCRIPTION

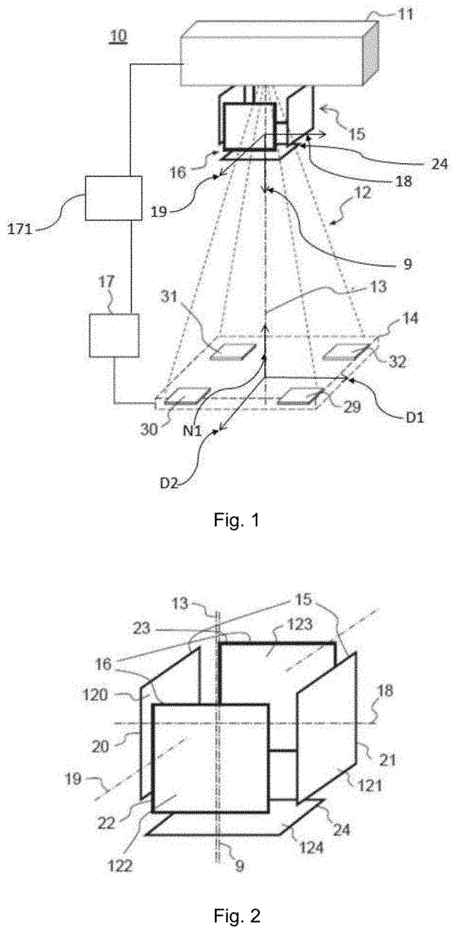

FIG. 1 shows one embodiment of a radiology assembly 10 according to the invention. The radiology assembly 10 comprises an x-ray tube 11 for generating a beam of x-rays 12 that is centered around a main emission direction 13. The radiology assembly 10 comprises a planar sensor 14 extending in a plane defined by a first direction D1 and by a second direction D2, which directions are substantially perpendicular to the main emission direction 13. The planar sensor 14 is intended to receive the x-rays 12. According to the invention, the radiology assembly comprises a first divided emitter 15 that is divided into two electromagnetic-field-emitting portions 20, 21 and is arranged so as to emit a first electromagnetic field in a main direction 18 that is substantially perpendicular to the main emission direction 13, each of the two emitting portions 20, 21 of the divided emitter 15 being positioned on one respective side of the beam of x-rays 12. Advantageously, the divided emitter 15 is securely fastened to the x-ray tube 11 for generating the beam of x-rays 12. In this configuration, the position of the x-ray tube 11 for generating the beam of x-rays 12 may be deduced from the main direction of the electromagnetic field emitted by the divided emitter 15.

Likewise, the radiology assembly may comprise a second divided emitter 16 that is divided into two electromagnetic-field-emitting portions 22, 23 and is arranged so as to emit a second electromagnetic field in a main direction that is substantially perpendicular to the main emission direction 13 and is secant to the main direction of the first electromagnetic field, each of the two emitting portions 22, 23 of the divided emitter 16 being positioned on one respective side of the beam of x-rays 12.

In other words, each divided emitter (15 for example) may be considered to be a pair of emitters 20, 21 the main faces of which are parallel to each other, each of the emitters being located on one respective side of the beam of x-rays 12. The pair of emitters 20, 21 (likewise for 22, 23) is equivalent to a virtual emitter that would be located between the two emitters 20, 21, in the beam of x-rays 12. Considering one divided emitter (i.e. one pair of emitters), the emitted electromagnetic field is equivalent to the electromagnetic field that would be emitted by the equivalent virtual emitter. This arrangement has the advantage of not obscuring the x-rays since the pair of emitters are located on either side of the beam of x-rays 12 and not in their beam. Moreover, this arrangement of the emitters has the advantage of not damaging the emitters. Specifically, an equivalent emitter placed in the beam of the x-rays would be damaged by the x-rays during its use. In the case of our invention, the emitters are not subjected to the x-rays and are therefore preserved from the material resistance point of view.

The radiology assembly 10 may further comprise a so-called planar electromagnetic-field emitter 24 arranged so as to emit a third electromagnetic field in a main direction 9 that is substantially parallel to the main emission direction 13. The so-called planar emitter 24 makes it possible to have an electromagnetic field that is parallel to the main emission direction 13.

The arrangement of the emitters as shown in FIG. 1 makes it possible to have electromagnetic fields the main directions of which are in three different axes perpendicular to each other. Since the emitters are securely fastened to the x-ray tube 11 for generating the beam of x-rays 12, the electromagnetic fields in the three axes make it possible to determine certain angular information making it possible to compare the different positions of the x-ray tube 11 and of the planar sensor 14. It may be noted that the three axes are not necessarily perpendicular to each other. The directions 18 and 19 may be secant and form any angle (between themselves and with the main emission direction 13). The relative position of the x-ray tube 11 with respect to the planar sensor 14 may also be determined.

The frequency of the first, second and third electromagnetic fields is subject to two constraints:
since the span and the power used in the divided emitters 15 and 16 and of the planar emitter 14 is relatively small, it is necessary to use a frequency that is sufficiently high in order to extract the signals from noise, all while obtaining a large range of emission for the first, second and third electromagnetic fields,
however, the presence of metallic objects in the environment of the radiology assembly 100 requires the use of low frequencies.

By way of example, the frequency of the first, second and third electromagnetic field may be a frequency comprised between 100 Hz and 10 kHz.

In addition, the first, second and third electromagnetic fields are emitted successively and in fixed and different orientations so as to avoid obtaining a rotating field and to avoid any interaction between the first, second and third electromagnetic fields. The three directions, preferably perpendicular, are addressed successively and independently with a field of fixed frequency, orientation and amplitude for a defined period.

The radiology assembly 10 comprises four electromagnetic-field sensors 29, 30, 31, 32. The four sensors 29, 30, 31, 32 may be integrated in the planar sensor 14. The sensors 29, 30, 31, 32 are intended to detect the electromagnetic fields emitted by the divided emitters 15 and 16 and by the so-called planar emitter 24 and to generate an electrical signal as a function of the detected electromagnetic fields. It should be noted that the radiology assembly may comprise fewer than four or more than four electromagnetic-field sensors.

The sensors 29, 30, 31, 32 are integrated in the planar sensor 14. They are installed in such a way that they do not interfere with the acquisition of the radiological image. They are for example placed behind the elements for detecting the radiological image with respect to the entrance face of the x-rays. They may have any position on the planar sensor 14. In this case, a corrective measure is necessary to determine the relative position of the x-ray tube 11 with respect to the planar sensor 14. If, in contrast, they are positioned perfectly symmetrically with respect to the center of the planar sensor, the perfect centering with respect to the x-ray tube 11 for generating the beam of x-rays 12 is obtained when the sensors 29, 30, 31, 32 have a perfectly balanced signal.

The radiology assembly 10 comprises a processing means 17 for processing the first, second, third electrical signal. Furthermore, the processing means 17 comprises a calculator able to determine an angle of alignment between the main emission direction 13 and a normal N1 of the planar sensor 14. The processing means 17 also comprises a calculator able to determine a first centering error between the main emission direction 18 of the first electromagnetic field and the first direction D1 of the planar sensor 14 and a second centering error between the main direction 19 of the second electromagnetic field and the second direction D2 of the planar sensor 14. The special feature of the radiology assembly according to the invention resides in its mode of alignment. Rather than considering the absolute position of the planar sensor as is done in the prior art, the invention performs alignment between the normal N1 of the planar sensor and the main emission direction 13 of the x-rays, then centering of the planar sensor around the normal N1, thus together with the main emission direction 13 of the x-rays.

The processing means 17 also comprises an estimator for estimating an angle of orientation between the main direction 18 of the first electromagnetic field and the first direction D1 of the planar sensor 14.

Furthermore, in order to ensure the robustness of the radiology assembly 10, the processing means 17 comprises means for distinguishing the generated electrical signals. Specifically, since each generated electrical signal induces a correction, it is necessary to correctly identify which electrical signal is captured at the sensors 29, 30, 31, 32 of the planar sensor 14.

The radiology assembly 10 also comprises a correcting means 171 for correcting the angle of alignment and the first and second centering errors. More specifically, upon receiving the angle of alignment and the first and second centering errors from the processing means 17, the correcting means 171 acts, for the case of a correction of the angle of alignment, by way of a first corrective movement on the x-ray tube 11 and acts, for the case of a correction of the first and/or second centering error, by way of the first corrective movement and/or a second corrective movement on the x-ray tube 11.

More specifically, the first corrective movement is a rotation of the x-ray tube 11 in one of the main directions 18 and 19 of the first and second electromagnetic field or a rotation of the x-ray tube 11 in the main emission direction 13. And the second corrective movement is a translation of the x-ray tube 11 in one of the main directions 18 and 19 of the first and second electromagnetic field. The first corrective movement like the second corrective movement may be carried out manually or be automated in connection with the correcting means 171.

Furthermore, in an analogous way, the correcting means 171 is able to correct the angle of orientation between the main direction 18 of the first electromagnetic field and the first direction D1 of the planar sensor 14 by applying the first corrective movement to the x-ray tube 11.

However, evaluation and correction of the angle of orientation remains optional with respect to the evaluation and correction of the angle of alignment and of the centering error. Specifically, in the case where the x-ray tube 11 for generating a beam of x-rays 12 and the planar sensor 14 are correctly aligned, that is to say when the angle of alignment is close to zero degrees, and where the x-ray tube 11 for generating a beam of x-rays 12 and the planar sensor 14 are correctly centered, that is to say when the centering error is close to zero, the angle of orientation is thus necessarily close to zero degrees. This angle of orientation is therefore a measure that makes it possible to confirm that the x-ray tube 11 for generating a beam of x-rays 12 and the planar sensor 14 are correctly aligned and correctly centered.

Finally, the processing means 17 and the correcting means 171 are, in one preferred embodiment, mechanically linked to the planar sensor 14. However, the processing means 17 and the correcting means 171 may also be mechanically linked to the x-ray tube 11.

When used in a radiological system, the typical operating distance is sufficiently large so that the magnetic field measured at the receiver (that is to say the planar sensor 14 in our case) can be considered to come from a magnetic dipole of moment \vec{M}.

In the Z-axis polar coordinate system of which the origin coincides with the center of the emitter block, the components Bᵣ, B_{θ} and B_{φ} of the magnetic field measured at a point M of spherical coordinates (ρ, θ, φ) in the frame of reference defined by the emitting source (in our case the x-ray tube 11) are thus:

$${B}_{r} = \frac{{μ}_{0}}{4 π} M \frac{2 cos θ}{{r}^{3}} ; \\ {B}_{θ} = \frac{{μ}_{0}}{4 π} M \frac{2 sin θ}{{r}^{3}} ; \\ {B}_{φ} = 0$$

Where μ0 is a fundamental constant, called vacuum magnetic permeability, r is the distance between the emitting source and the point M and θ is the angle of alignment close to the alignment.

The components of the fields Bₓ, B_{y}, B_{z}, measured at the point M and denoted \vec{Bₘₓ}, \vec{B_{my}} and \vec{B_{mz}} are therefore:

$$\vec{{B}_{m x}} = \vec{{B}_{( mx , 0 , 0 )}} = \frac{{μ}_{0} {m}_{x}}{4 π {r}^{3}} [ \begin{matrix}2 sin θ cos φ \cdot \vec{{μ}_{r}} \\ - cos θ cos φ \cdot \vec{{μ}_{θ}} \\ sin φ \cdot \vec{{μ}_{φ}}\end{matrix} \vec{{B}_{m y}} = \vec{{B}_{( 0 , my , 0 )}} = \frac{{μ}_{0} {m}_{y}}{4 π {r}^{3}} [ \begin{matrix}2 sin θ sin φ \cdot \vec{{μ}_{r}} \\ - cos θ sin φ \cdot \vec{{μ}_{θ}} \\ - cos φ \cdot \vec{{μ}_{φ}}\end{matrix} \vec{{B}_{m z}} = \vec{{B}_{( 0 , 0 , mz )}} = \frac{{μ}_{0} {m}_{z}}{4 π {r}^{3}} [ \begin{matrix}2 cos θ \cdot \vec{{μ}_{r}} \\ sin θ \cdot \vec{{μ}_{θ}} \\ 0\end{matrix}$$

Where φ is the angle of longitude between the emitting source and the point M.

Since the fields \vec{Bₘₓ}, \vec{B_{my}} and \vec{B_{mz}} are measured, it is possible to calculate the scalar products close to the alignment (θ≈0)

$$\frac{\vec{{B}_{m x}} \cdot \vec{{B}_{mz}}}{‖ \vec{{B}_{m x}} ‖ \cdot ‖ \vec{{B}_{mz}} ‖} = \frac{3}{2} \cdot θ \cdot cos φ + o ( θ ) \\ And \\ \frac{\vec{{B}_{my}} \cdot \vec{{B}_{mz}}}{‖ \vec{{B}_{my}} ‖ \cdot ‖ \vec{{B}_{mz}} ‖} = \frac{3}{2} \cdot θ \cdot sin φ + o ( θ )$$

By virtue of the simplifications and approximations possible in an alignment strategy, it is thus possible to estimate the alignment deviation with a precision that is all the better the smaller this deviation is. This deviation corresponds to a relative rotation position between the emitter and the detector. It is corrected by means of the first corrective movement by applying a rotation to the detector (the planar sensor 14) in the main emission direction 13 or a reverse rotation to the emitting source (the x-ray tube 11 in our case) in the main emission direction 13. This simplification also has the advantage of resulting in a calculation that is relatively simple and therefore not very costly in terms of computing time and power.

There is a first interaction between the translation in the main direction 18 of the first electromagnetic field and the rotation in the main direction 19 of the second electromagnetic field and a similar second interaction between the translation in the main direction 19 of the second electromagnetic field and the rotation in the main direction 18 of the first electromagnetic field. The first interaction is described in that a translation in the main direction 18 of the first electromagnetic field between the detector (the planar sensor 14) and the emitting source (the x-ray tube 11) in the main direction 18 of the first electromagnetic field involves a rotation of the field measured at the detector (the planar sensor 14) that is similar and opposite to the application of a rotation in the main direction 19 of the second electromagnetic field between the detector (the planar sensor 14) and the emitting source (the x-ray tube 11).

The interaction is resolved by using an inclinometer at the detector (the planar sensor 14) in order to evaluate the alignment with respect to the emitting source (the x-ray tube 11). Knowledge of the angle of alignment makes it possible to apply a relative rotation between the detector (the planar sensor 14) and the emitting source (the x-ray tube 11) in order to obtain alignment between the planar sensor 14 and the x-ray tube 11.

Once the horizontal planes of the detector (the plane formed by the first direction D1 and the second direction D2 of the planar sensor 14) and of the emitting source (the plane formed by the main directions 18 and 19 of the x-ray tube 11) are aligned, the angle measured at the fields arises only from displacements respectively linked to the first centering error in the main direction 18 and to the second centering error in the main direction 19. Through calculations and estimations similar to the development hereinabove, it is also possible to measure the rotation of the magnetic field in the main directions 18 and 19 and to deduce therefrom the approximated values of correction of the first and second centering errors which define the position of the radiological detector with respect to the emitting source.

The distance, in the main emission direction 13, of the positioning of the detector (the planar sensor 14) with respect to the emitting source (the x-ray tube 11) when the two are aligned is not a measure to be fixed at a precise value. This distance must simply be comprised between a minimum value and a maximum value which are characteristic of the anti-scatter grid. This value may nevertheless be estimated by the measure of the module of the electromagnetic field at the center of the detector (planar sensor 14) by taking the average of the values measured at the sensors 29, 30, 31, 32 and by correlating it with a calibration of the module of the induction as a function of the distance between the detector (the planar sensor 14) and the emitting source (the x-ray tube 11).

Since all of these formulae become very simple close to the alignment of the planar sensor 14 and of the x-ray tube 11, it is possible to calculate the positions and angles with a precision that is all the better the closer the detector (the planar sensor 14) is to the target position. These approximations make it possible to obtain a sufficient precision locally whereas solving the inverse problem would become very complicated if this same precision was sought in the entirety of the field of use, necessitating for example recourse to methods and algorithms such as Kalman filters, likewise costly in terms of computing time and therefore disadvantageous for the response time of the system or for the complexity of the calculator.

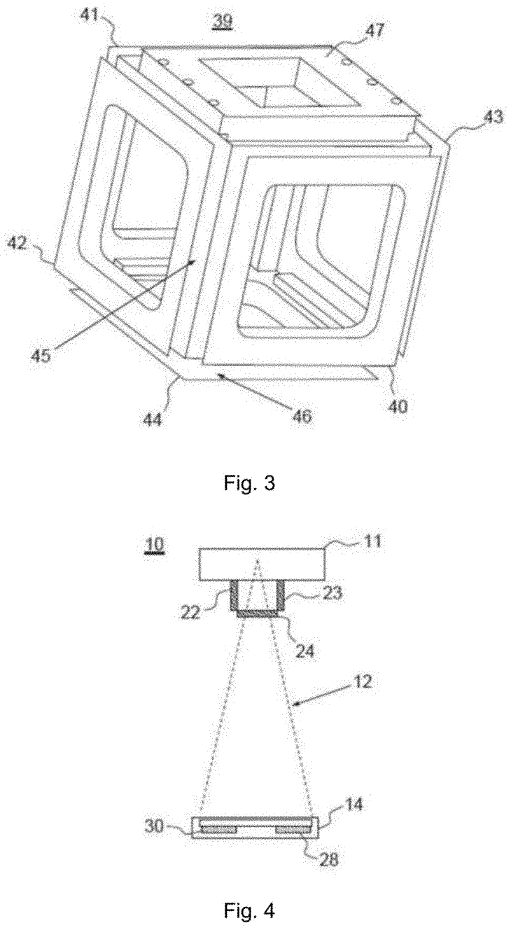

FIG. 2 shows an example of an arrangement of the electromagnetic-field emitters 15 and 16 according to the invention. In FIG. 2, the radiology assembly comprises two divided emitters 15, 16 that are divided into two electromagnetic-field-emitting portions 20, 21 and 22, 23. The first divided emitter 15 that is divided into two electromagnetic-field-emitting portions 20, 21 is arranged so as to emit a first electromagnetic field in a main direction 18 that is substantially perpendicular to the main emission direction 13. Each of the two emitting portions 20, 21 of the divided emitter 15 is positioned on one respective side of the beam of x-rays 12. Likewise, the second divided emitter 16 that is divided into two electromagnetic-field-emitting portions 22, 23 is arranged so as to emit a second electromagnetic field in a main direction 19 that is substantially perpendicular to the main emission direction 13 and substantially perpendicular to the main direction 18 of the first electromagnetic field. Each of the two emitting portions 22, 23 of the divided emitter 16 is positioned on one respective side of the beam of x-rays 12.

The divided emitters 15 and 16 and the so-called planar emitter 24 may be, by way of example, coils or solenoids. More specifically, each of the two emitting portions 20, 21 of the divided emitter 15 and each of the two emitting portions 22, 23 of the divided emitter 16 comprises at least one winding through which a current may flow. Furthermore, in the manner, the so-called planar emitter 24 comprises at least one winding through which a current may flow.

If the surface represented by the winding of each of the emitting portions 20, 21 and 22, 23 is now considered, it may be noted that a surface 120 of the emitting portion 20 is substantially parallel to a surface 121 of the emitting portion 21. Furthermore, the electromagnetic field emitted by the divided emitter 15 has a main direction 18 that is perpendicular to the surfaces 120 and 121. On the same principle, a surface 122 of the emitting portion 22 is substantially parallel to a surface 123 of the emitting portion 23. Furthermore, the electromagnetic field emitted by the divided emitter 16 has a main direction 19 that is perpendicular to the surfaces 122 and 123. Advantageously, the surfaces 120 and 121 are perpendicular to the surfaces 122 and 123. In addition to being secant, the main directions 18 and 19 are then substantially perpendicular to each other. This arrangement is in particular advantageous if the x-ray tube 11 for generating the beam of x-rays 12 has an emission flux of square shape. Thus, the flux of x-rays 12 is emitted in the main emission direction 13, between the surfaces 120, 121, 122, 123, without intersecting the emitters 15, 16 (and therefore without damaging them) and without being obscured since the emitters 15, 16 are not located in the flux of x-rays 12.

This configuration of the divided emitters 15 and 16 makes it possible to observe that the main emission direction 13 of the beam of x-rays 12 is positioned between the at least one winding of the divided emitter 15 and of the divided emitter 16 and makes it possible to ensure that each of the pairs of emitters 20, 21 and 22, 23, the respective surfaces 120, 121 and 122, 123 of which are parallel to each other, is equivalent to a virtual emitter located at the center of the surfaces 120, 121, 122, 123 of the emitters 15, 16, level with the main emission direction 13 of the x-rays, whereas it would be impossible to place a single emitter at the center since the center is occupied by the beam of x-rays. Thus, the emitters may emit, in an off-centered position, an electromagnetic field equivalent to an electromagnetic field emitted in a centered position, without obscuring the x-rays emitted by the x-ray tube 11. In addition, the at least one winding of the divided emitters 15 and 16 and of the so-called planar emitter 24 may be of square shape or of rectangular shape or else of circular shape.

In the same manner, a surface represented by the winding of the so-called planer emitter 24 may be interpreted as the surface 124 of the so-called planar emitter 24. This surface 124 of the so-called planar emitter 24 is substantially perpendicular to the surfaces 120, 121, 122, 123. Unlike the divided emitters 15 and 16, the flux of x-rays 12 may pass through the so-called planar emitter 24 at the winding. The flux of x-rays 12 is not obscured by the so-called planar emitter 24 due to the fact that it passes though it through the winding or windings.

The arrangement of the emitters as shown in FIG. 2 makes it possible to have electromagnetic fields the main directions of which are in three different axes perpendicular to each other. Since the divided emitters 15 and 16 and the so-called planar emitter 24 are securely fastened to the x-ray tube 11 for generating the beam of x-rays 12, the electromagnetic fields in the three axes make it possible to determine certain angular information such as the angle of alignment, the first and second centering errors or else the angle of orientation of the x-ray tube 11 for generating the beam of x-rays 12 with respect to the planar sensor 14.

It may be noted that the three axes are not necessarily perpendicular to each other. The directions 18 and 19 may be secant and form any angle (between themselves and with the main emission direction 13). More broadly, the electromagnetic fields in the three axes make it possible to determine the position of the x-ray tube 11 for generating the beam of x-rays 12 with respect to the planar sensor 14.

In FIG. 2, the emitters are three in number (15, 16, 24, i.e. 4 emitting portions 20, 21, 22, 23 and one emitter 24) and positioned so as to form a rectangular parallelepiped. Nevertheless, it is entirely envisionable for there to be more than three emitters, each being positioned on one face of a polyhedron the number of faces of which would correspond to the number of emitting portions and emitters used. A larger number of emitters adds to the precision of the evaluation of the angle of alignment, of the first and second centering errors and, optionally, of the angle of orientation of the x-ray tube 11 with respect to the planar sensor 14. Nevertheless, this larger number increases production costs and increases the complexity of the processing of the signals. Three emitters, as in FIG. 2, represent a very good compromise between precision of the evaluation of the angular information and signal processing complexity.

FIG. 3 shows an example of a holder 39 of the electromagnetic-field emitters. Corresponding to the configuration of FIG. 2, the holder 39 has faces 40, 41, 42, 43, 44 that are substantially perpendicular to one another. The face 42 has a groove 45 that is able to receive the emitting portion 22. Likewise, the face 44 has a groove 46 that is able to receive the emitter 24. The same goes for each of the faces. The holder 39 comprises an intermediate element 47, which is substantially perpendicular to the faces 40, 41, 42, 43 and substantially parallel to the face 44. The intermediate element 47 is a fastening means making it possible to securely fasten the holder 39 (and therefore the emitters 15, 16, 24) to the x-ray tube 11 for generating the beam of x-rays 12.

In the case of a configuration with a plurality of other emitters, the holder 39 then has another three-dimensional geometric shape having planar faces, each planar face having a groove arranged to house one emitter. There may be other arrangements, in particular the case in which the emitters are produced on a printed circuit board. In this case, flat coils may be fixed to the faces of a collimator, acting as a rigid and mobile framing structure of the x-ray tube 11 for generating the beam of x-rays 12. Thus, the two emitting portions 20, 21 and 22, 23 of the first and second divided emitter 15 and 16 and the so-called planar emitter 24 are flat coils, thus reducing the overall size of the system.

It may also be envisaged that the flat coils replace the faces of the collimator or be directly integrated in the collimator.

By virtue of the geometry presented in FIGS. 2 and 3, the pairs of surfaces that are parallel to each other (120 and 121, 122 and 123) and the placement of the emitters in the grooves that are provided for this purpose means that the left and right windings (of faces 42 and 43) and/or front and back windings (of faces 40 and 41) are very symmetric, making it possible to have a magnetic field that is perfectly centered on the center of the geometric shape without hindering the passage of the x-rays. It is not necessary to have a plurality of windings in the grooves of the lateral faces, the bottom winding, that is to say that of the face 44 in the groove 46, being sufficient for symmetry.

In other words, each divided emitter (15, 16) is divided into two electromagnetic-field-emitting portions (20, 21; 22, 23) that are configured to generate an electromagnetic field that is perfectly centered between the two faces that the emitting portions form. The two emitting portions each have a surface, their two surfaces being parallel to each other.

FIG. 4 shows a cross-sectional view of the radiology assembly 10 according to the invention. As said previously, in FIG. 1, the sensors 29, 30, 31, 32 are integrated in the planar sensor 14. They are installed in such a way that they do not interfere with the acquisition of the radiological image. They are for example placed behind the elements for detecting the radiological image with respect to the entrance face of the x-rays.

The electromagnetic-field sensors 29, 30, 31, 32 may for example be coils, magnetometers, magnetoresistors, anisotropic magnetoresistors, magneto-transistors, magneto-diodes, fluxgates or Hall-effect sensors. Furthermore, the planar sensor 14 and the x-ray tube 11 for generating the beam of x-rays 12 may comprise at least one inclinometer. Specifically, inclinometers placed at the x-ray tube 11 and at the planar sensor 14 make it possible to evaluate the acceleration of gravity on the emitting portion, that is to say at the x-ray tube 11, and on the receiving portion, at the planar sensor 14. This acceleration, which gives an absolute vector and is normally identical for the emitting and receiving portions, must then be projected differently depending on the observable deviation from the alignment, the centering and the orientation of the x-ray tube 11 with respect to the planar sensor 14.

If the x-ray tube 11 and the planar sensor 14 are parallel, that is to say that the plane formed by the main directions 15 and 16 of the x-ray tube 11 is parallel to the plane formed by the first direction D1 and by the second direction D2 of the planar sensor 14, the absolute vector of the x-ray tube 11 is then collinear with the absolute vector of the planar sensor 14.

Otherwise, the angle formed between the absolute vector of the x-ray tube 11 and the absolute vector of the planar sensor 14 corresponds to an inclination between the plane formed by the main directions 15 and 16 of the x-ray tube 11 and the plane formed by the first direction D1 and by the second direction D2 of the planar sensor 14 and therefore to a misalignment between the x-ray tube 11 and the planar sensor 14.

Each of the electromagnetic-field sensors 29, 30, 31, 32 may comprise an amplifying and filtering electronic circuit (not shown in the figure) that is intended to process the electrical signal generated by each of the sensors 29, 30, 31, 32. Each sensor 29, 30, 31, 32 detects an electromagnetic field and generates an electrical signal depending on the amplitude of the detected electromagnetic field. The generated electrical signal is processed by the amplifying and filtering electronic circuit.

Depending on the type of sensor used, at any given time, each sensor 29, 30, 31, 32 may generate one or more pieces of information. If the sensor is single-axis, it generates a single piece of information. If the sensor is multi-axis, it generates a plurality of pieces of information. The use of multi-axis sensors makes it possible to know the amplitude of the electromagnetic field and its orientation.

In our configuration, if the sensors are single-axis sensors, for a given position of the planar sensor 14, twelve pieces of information are generated. If the sensors are tri-axis sensors, then thirty-six pieces of information are generated.

The detected signals are digitized and transmitted to the calculator of the processing means 17, shown in FIG. 1, which processes the pieces of angular information such as the angle of inclination, the first and second centering error or else the angle of orientation of the planar sensor 14 with respect to the x-ray tube 11 for generating the beam of x-rays 12. The pieces of information coming from the sensors 29, 30, 31, 32 are then transmitted in digital form. They may thus be transmitted either over a wired link or over a wireless link.

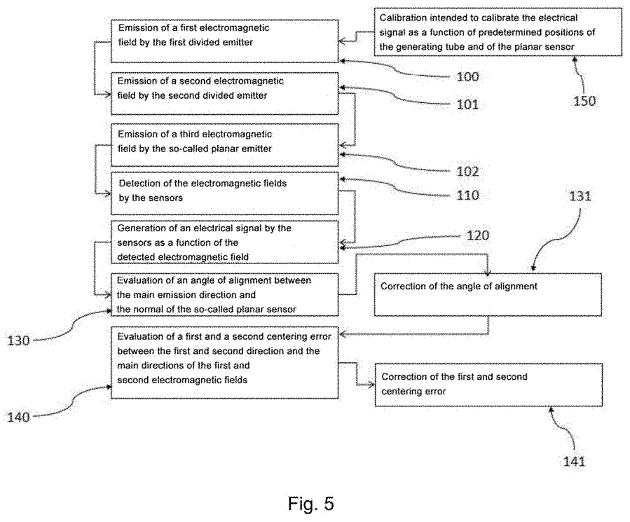

FIG. 5 schematically shows the steps of an aligning method according to the invention. The method for aligning a radiology assembly 10 according to the invention includes the following steps:
Emission by the first divided emitter 15 of the first electromagnetic field (step 100) in the main direction 18 that is substantially perpendicular to the main emission direction 13,
Emission by the second emitter of the second electromagnetic field (step 101) in the main direction 19 that is substantially perpendicular to the main emission direction 13,
Emission by the so-called planar emitter of a third electromagnetic field (step 102) in a main direction 9 that is substantially parallel to the main emission direction 13,
Detection by the sensors of the electromagnetic fields emitted alternately in their main direction by the first emitter 15, the second emitter 16 and the so-called planar emitter 24 (step 110),
Generation of the first, second, third electrical signals by the sensors 29, 30, 31, 32 depending on the first, second, third detected electromagnetic fields (step 120),
Evaluation of the angle of alignment (step 130) between the main emission direction 13 and a normal of the planar sensor 14,
Correction of the angle of alignment (step 131) between the main emission direction 13 and a normal N1 of the planar sensor 14 by applying the first corrective movement,
Evaluation of the first centering error between the main emission direction 18 of the first electromagnetic field and the first direction D1 of the planar sensor 14, and of the second centering error between the main emission direction 19 of the second electromagnetic field and the second direction D2 of the planar sensor 14 (step 140),
Correction of the first and the second centering error (step 141) by applying the first corrective movement and/or the second corrective movement.

Optionally, repetition of the preceding steps until the angle of alignment is less than a predefined threshold angle of alignment and/or until the first centering error and the second centering error are less than a predefined threshold first centering error and than a predefined threshold second centering error.

Specifically, in the case of a radiology assembly 10 comprising a plurality of emitters 15, 16, 24, the processing means 17 must comprise means for distinguishing the generated electrical signals as a function of the first, second and third electromagnetic field detected.

The step 130 of evaluation of the angle of alignment between the main emission direction 13 and the normal of the planar sensor 14 is carried out by processing the first, second and third electrical signals by means of, for example, the amplifying and filtering electronic circuit stated above. Thus, by means of a calculator, the angle of alignment is evaluated and analyzed in order to better know the difference in alignment between the x-ray tube 11 and the planar sensor 14. Specifically, the angle of alignment makes it possible to bring to light a parallelism between the plane formed by the main directions 18 and 19 of the first and second electromagnetic fields at the x-ray tube 11 and the plane formed by the first and the second direction D1 and D2 of the planar sensor 14.

Thus, the step 131 of correction of the angle of alignment makes it possible to achieve parallelism between the plane of the x-ray tube 11, which is perpendicular to the x-ray beam 13, and the plane of the planar sensor 14 stated above. To do this, the first corrective movement is applied so as to obtain a rotation in one of the main directions 18, 19 of the first or second electromagnetic field. In this manner, it is ensured that the beam of x-rays 12 is correctly aligned facing the planar sensor 14 and that irradiating outside of the planar sensor 14 is avoided.

In the same manner, the step 140 of evaluation of the first centering error between the main emission direction 18 of the first electromagnetic field and the first direction D1 of the planar sensor 14, and of the second centering error between the main emission direction 19 of the second electromagnetic field and the second direction D2 of the planar sensor 14 is carried out by processing the first, second and third electrical signals by means of, for example, the amplifying and filtering electronic circuit stated above. The evaluation and analysis of the first and second centering error makes it possible to bring to light a potential lack of centering between the x-ray tube 11 and the planar sensor 14. This then results in irradiation of the beam of x-rays 12 outside of the area of the planar sensor 14, this not being optimal.

The step 141 of correction of the first centering error and the second centering error then makes it possible to refocus the x-ray tube 11 with respect to the planar sensor 14. To do this, the x-ray tube 11 is subjected to a rotation in the main emission direction 13 until the main direction 18 of the first electromagnetic field of the x-ray tube 11 is parallel to the first direction D1 of the planar sensor 14 and until the main direction 19 of the second electromagnetic field is parallel to the second direction D2 of the planar sensor 14 by applying the first corrective movement. Thus, the plane formed by the main directions 18 and 19 of the first and second electromagnetic fields at the x-ray tube 11 and the plane formed by the first and the second direction D1 and D2 of the planar sensor 14 are then collinear.

Following this, the x-ray tube 11 is subjected to a translation in the main direction 18 of the first electromagnetic field and/or in the main direction 19 of the second electromagnetic field by applying the second corrective movement until the projection of the main direction 18 of the first electromagnetic field and of the main direction 19 of the second electromagnetic field on the plane formed by the first direction D1 and the second direction D2 of the planar sensor 14 are respectively the first direction D1 and the second direction D2 of the planar sensor 14. In this manner, the x-ray tube 11 is aligned with the planar sensor 14, this optimizing the irradiation.

The aligning method according to the invention may include, beforehand, a calibration step 150 intended to calibrate the electrical signal as a function of predetermined positions of the x-ray tube 11 and of the planar sensor 14. During this step, the angular information mentioned above is stored and then used in order to determine corrective terms which will be taken into account during the following steps.

The aligning method according to the invention may include, following the step 141, a step of evaluation of an angle of orientation between the main direction 18 of the first electromagnetic field and the first direction D1 of the planar sensor 14 carried out by processing the first, second and third electrical signals by means of, for example, the amplifying and filtering electronic circuit stated above. This step makes it possible to validate correct parallelism between the main direction 18 of the first electromagnetic field and the first direction D1 of the planar sensor 14. Specifically, although this parallelism is verified during the step 141, evaluation of the angle of orientation makes it possible to provide an additional verification, this increasing the precision and the robustness of the aligning method according to the invention.

Thus, the processing means 17 for processing the first, second, third electrical signal of the radiology assembly 10, shown in FIG. 1, may comprise an estimator for estimating the angle of orientation optionally in addition to the estimation of the angle of alignment of the first and second centering error of the x-ray tube 11 with respect to the planar sensor 14.

In addition, following this step of evaluation of the angle of orientation, an additional step of correction of the angle of orientation may be introduced in order to correct the parallelism between the main direction 18 of the first electromagnetic field and the first direction D1 of the planar sensor 14 by applying the first corrective movement in the main emission direction 13. Thus, this step of evaluation and correction of the angle of orientation is optional but increases the robustness and the precision of the aligning method.

Furthermore, the aligning method may comprise a step of validation of the alignment of the x-ray tube 11 and of the planar sensor 14 following the step 131. This step of validation of the alignment makes it possible to judge the correct alignment of the two elements mentioned above. To do this, the angle of alignment, corrected during the step 131, is compared with a threshold angle of alignment, which may be, for example, from 1° à 2°. In this manner, if the validation of the alignment is not conclusive, that is to say if the angle of alignment, corrected during the step 131, remains greater than the threshold angle of alignment, the angle of alignment may then be subjected to a new correction of the angle of alignment (step 131) between the main emission direction 13 and the normal N1 of the planar sensor 14 by additionally applying the first corrective movement.

In the same manner, the aligning method may comprise a step of validation of the centering of the x-ray tube 11 and of the planar sensor 14 following the step 141. This step of validation of the centering makes it possible to judge the correct centering of the two elements mentioned above. To do this, the first centering error and the second centering error, corrected during the step 141, are respectively compared with a threshold first centering error and with a threshold second centering error, which may be, for example, about 2 centimeters to 5 centimeters. In this manner, if the validation of the centering is not conclusive, that is to say if the first centering error and the second centering error, corrected during the step 141, remain greater than the threshold first centering error and than the threshold second centering error, the first centering error and the second centering error may then be subjected to a new correction of the first centering error and the second centering error (step 141 by additionally applying by applying the first corrective movement and/or the second corrective movement).

Thus, it is possible to observe a repetition of the preceding steps until the angle of alignment is less than the threshold angle of alignment and/or until the first centering error and the second centering error are less than the threshold first centering error and than the threshold second centering error.

Lastly, the emitters 15, 16, 24 are supplied with power by the electrical signals at different times or simultaneously at different frequencies or simultaneously in phase offset so as to differentiate the emitted electromagnetic fields.

In other words, the first divided emitter 15 and the second divided emitter 16 may be supplied with power at different times or simultaneously at a different frequency or in phase offset. The fact of supplying the divided emitters with power at different times or simultaneously at a different frequency or in phase offset is one means for distinguishing the generated electrical signals.

Likewise, the so-called planar emitter 24 and the first divided emitter 15 and the second divided emitter 16 may be supplied with power at different times or simultaneously at different frequencies or in phase offset.

The invention described has several advantages over the existing solutions:
The combination of an emitting source/low-frequency magnetic receiver assembly associated with synchronous detection makes it possible to reduce the sensitivity of the aligning system with respect to external interference such as:
electromagnetic interference, including the magnetic field of the Earth,
the presence of objects between the emitting source and the receiver, these objects being opaque to high-frequency radiation, including light,
The principle of detecting and calculating locally allows the resolution of the inverse magnetic problem in the area in which precision of the alignment is sought rather than precision of uniform positioning everywhere which is not necessary and would be more costly in terms of computing time and power,
The search for the alignment allows operation with increased distances between the source and the receiver,
The system may operate inside a building in an environment affected by interference,
The invention may be easily implemented by adding an emitting system that is securely fastened to the x-ray source and its collimator and by adding small-sized sensors integrated in the electronics of the detector,
The calculated information may easily be transmitted to the radiological system in order to automatically align the tube with the detector.

The main innovation is a method making it possible to simply resolve the problem of alignment between an emitting source and a receiver in an iterative manner without recourse to complex calculations, estimations and algorithms.

This method is based on:
A renewal with a rate lower than the second of the tri-axial measurement of the magnetic fields emitted in three orthogonal directions,
A simplified method for calculating the position and the orientation that is all the more precise the closer the axes are to the alignment, this method making it possible to resolve the inverse magnetic problem through simplified calculations that do not require a complex algorithm or high computing power,
The possibility to combine the original magnetic information with the information provided by inertial sensors in order to improve the precision of the correction,
The possibility to use the result of the calculations in order to facilitate manual alignment or in order to send control signals to an automatic alignment device.

By favoring an alignment strategy over precise positioning at all points in space, it is possible to reduce the required computing time and power. The delay time may thus be minimized and the measurement may be renewed with a higher frequency. Consequently, displacements will be more precise and less jerky.

1. A radiology assembly comprising:
an x-ray tube for generating a beam of x-rays that is centered around a main emission direction,
a planar sensor extending in a plane defined by a first direction (D1) and by a second direction (D2), which directions are substantially perpendicular to the main x-ray emission direction, the sensor being intended to receive the x-rays, comprising:
a first divided emitter that is divided into two electromagnetic-field-emitting portions and is arranged so as to emit a first electromagnetic field in a main direction that is substantially perpendicular to the main emission direction, each of the two emitting portions of the divided emitter being positioned on one respective side of the beam of x-rays,
a second divided emitter that is divided into two electromagnetic-field-emitting portions and is arranged so as to emit a second electromagnetic field in a main direction that is substantially perpendicular to the main emission direction and is secant to the main direction of the first electromagnetic field, each of the two portions of the divided emitter being positioned on one respective side of the beam of x-rays,
a so-called planar electromagnetic-field emitter, the so-called planar emitter being a coil composed of windings, the so-called planar emitter being arranged so as to emit a third electromagnetic field in a main direction that is substantially parallel to the main emission direction of the beam of x-rays, the windings being passed through by the main emission direction,
electromagnetic-field sensors that are securely fastened to the planar sensor and are able to detect the first, second and third electromagnetic fields emitted alternately in their main direction by the first emitter, the second emitter and the so-called planar emitter and to generate a first, second, third electrical signal depending on the detected electromagnetic fields,
a processing means for processing the first, second and third electrical signals that is intended to determine an angle of alignment between the main emission direction and a normal (N1) of the planar sensor, to determine a first centering error between the main emission direction of the first electromagnetic field and the first direction (D1) of the planar sensor, to determine a second centering error between the main emission direction of the second electromagnetic field and the second direction (D2) of the planar sensor, a correcting means for correcting the angle of alignment by applying a first corrective movement to the x-ray tube and first and second centering errors by applying the first corrective movement and/or a second corrective movement to the x-ray tube.

2. The radiology assembly as claimed in claim 1, wherein the processing means comprises means for distinguishing the generated electrical signals.

3. The radiology assembly as claimed in claim 1, wherein the processing means for processing the first, second and third electrical signals comprises an estimator for estimating an angle of orientation between the main direction of the first electromagnetic field and the first direction (D1) of the planar sensor.

4. The radiology assembly as claimed in claim 1, wherein each of the two emitting portions of the first and second divided emitter comprises at least one winding and in that the main emission direction of the beam of x-rays is positioned between the at least one winding of the first and second divided emitter.

5. The radiology assembly as claimed in claim 1, wherein the so-called planar emitter comprises at least one winding that is passed through by the main emission direction of the beam of x-rays.

6. The radiology assembly as claimed in claim 1, wherein the two emitting portions of the first and second divided emitter and the so-called planar emitter are flat coils.

7. The radiology assembly as claimed in claim 1, wherein the first corrective movement is a rotation of the x-ray tube in one of the main directions and/or a rotation of the x-ray tube in the main emission direction and wherein the second corrective movement is a translation of the x-ray tube in one of the main directions.

8. The radiology assembly as claimed in claim 1, wherein the planar sensor comprises at least one inclinometer.

9. The radiology assembly as claimed in claim 1, wherein the processing means and the correcting means are mechanically linked to the planar sensor.

10. The radiology assembly as claimed in claim 1, wherein the processing means and the correcting means are mechanically linked to the x-ray tube.

11. A method for aligning a radiology assembly as claimed in claim 1, including the following steps:
emission by the first divided emitter of the first electromagnetic field (step 100) in the main direction that is substantially perpendicular to the main emission direction,
emission by the second emitter of the second electromagnetic field in the main direction that is substantially perpendicular to the main emission direction,
emission by the so-called planar emitter of a third electromagnetic field in a main direction that is substantially parallel to the main emission direction,
detection by the sensors of the electromagnetic fields emitted alternately in their main direction by the first emitter, the second emitter and the so-called planar emitter (step 110),
generation of the first, second, third electrical signals by the sensors depending on the first, second, third detected electromagnetic fields (step 120),
evaluation of the angle of alignment between the main emission direction and a normal of the planar sensor,
correction of the angle of alignment between the main emission direction and a normal of the planar sensor by applying the first corrective movement,
evaluation of the first centering error between the main emission direction of the first electromagnetic field and the first direction (D1) of the planar sensor, and of the second centering error between the main emission direction of the second electromagnetic field and the second direction (D2) of the planar sensor,
correction of the first and the second centering error by applying the second corrective movement,
optionally, repetition of the preceding steps until the angle of alignment is less than a predefined threshold angle of alignment and/or until the first centering error and the second centering error are less than a predefined threshold first centering error and than a predefined threshold second centering error.

12. The aligning method as claimed in claim 11, including, beforehand, a calibration step intended to calibrate the electrical signal as a function of predetermined positions of the x-ray tube and of the planar sensor.

13. The aligning method as claimed in claim 11, wherein the emission by the emitters of the electromagnetic fields includes a step of supplying the emitters with power, and in that the emitters are supplied with power at different instants or simultaneously at different frequencies or simultaneously in phase offset so as to differentiate the emitted electromagnetic fields.

14. The aligning method as claimed in claim 11, comprising a step of evaluation of the angle of orientation between the main direction of the first electromagnetic field and the first direction (D1) of the planar sensor following the step of correction of the centering error and comprising a step of correction of the angle of orientation between the main direction of the first electromagnetic field and the first direction (D1) of the planar sensor following the step of evaluation of the angle of orientation.
